(19) United States
(12) Patent Application Publication
(10) Pub. No.: US 2021/0353496 A1
(43) Pub. Date: Nov. 18, 2021

(54) LIMB COMPRESSION DEVICE

(71) Applicant: OSTEOSYS CO., LTD., Seoul (KR)

(72) Inventors: Young Bok AHN, Seoul (KR); Won Hee LEE, Anyang-si Gyeonggi-do (KR); Jae Jin KIM, Seoul (KR); Jung Tae KIM, Seoul (KR)

(73) Assignee: OSTEOSYS CO., LTD., Seoul (KR)

(21) Appl. No.: 16/622,216

(22) PCT Filed: Jul. 10, 2018
(86) PCT No.: PCT/KR2018/007775

§ 371 Date: Dec. 12, 2019

(30) Foreign Application Priority Data
Jul. 9, 2018 (KR) 10-2018-0079260

(51) Int. Cl. A61H 11/00

(52) U.S. Cl. CPC A61H 11/00; A61H 2201/1215; A61H 2201/50; A61H 2230/505; A61H 2230/305; A61H 2230/255; A61H 2209/00

(57) ABSTRACT

The present invention provides a limb compression device, including: a main body having a first surface where a support portion wrapping around a portion of a limb is provided, and a second surface where a fixing shaft fixing a cuff wrapping around a remaining portion of the limb is mounted; a drive motor coupled to the main body; a reduction gear unit coupled to a drive shaft of the drive motor; a cuff fixing drum fixing the cuff, and having a first end coupled to the reduction gear unit and a second end coupled to the main body such that the cuff fixing drum winds and unwinds the cuff; and the cuff including a first end fixed to the cuff fixing drum and a second end wound around the fixing shaft via the support portion of the main body so as to wrap around the remaining portion of the limb.

### TECHNICAL FIELD

The present invention generally relates to a limb compression device. More particularly, the present invention relates to a limb compression device that wraps around a limb of a subject, and compresses or decompresses the wrapped limb when driven in response to a control signal of a controller receiving a signal from a biosignal sensing device or a user input device, thus improving blood flow or alleviating edema.

### BACKGROUND ART

As well known in the art, a limb compression device is a device that improves blood flow or relieves edema by periodically pressurizing or releasing a cuff wrapped around a person's arm or leg (hereafter referred to as limb).

A limb compression device in the related art is configured to perform compression therapy, whereby pneumatic pressure generated by driving of a pneumatic pressure generator is supplied to each air pocket of a cuff to compress the body contact area, thus reducing the cross-sectional area of blood vessels and thus increasing the velocity of blood flow and helping blood circulation. Alternatively, the limb compression device is configured to perform massage by repeatedly compressing or releasing the body contact area as the volume of an air layer that inflates or deflates increases or decreases.

However, the limb compression device using pneumatic pressure in the related art is limited in compression intensity of the cuff because the pneumatic pressure injected into the air pocket is used to inflate the cuff to compress the body contact area.

Therefore, the limb compression device using pneumatic pressure can be used for the purpose of prevention of deep vein thrombosis (DVT) and varicose veins, blood flow improvement, massage, and vibration, but is insufficient for the purpose of medical rehabilitation (external counterpulsation, ECP) of cardiovascular diseases, such as angina pectoris, heart failure, myocardial infarction, and the like that require compression intensity.

### DISCLOSURE

### Technical Problem

Accordingly, the present invention has been made keeping in mind the above problems occurring in the related art, and

an objective of the present invention is to provide a limb compression device that is used not only for purposes of treatment and prevention of diseases caused by problems in blood circulation, such as deep vein thrombosis, angina pectoris, heart failure, myocardial infarction, and the like that require compression intensity, but also for purposes of massage and vibration by controlling compression intensity, timing, and repetition frequency.

Another objective of the present invention is to provide a limb compression device that realizes a compact design through a simplified configuration and an efficient layout structure, thus increasing portability, while increasing convenience of use by not restricting a wearer's activity.

### Technical Solution

In order to accomplish the above objectives, according to one aspect of the present invention, there is provided a limb compression device, including: a main body having a first surface where a support portion wrapping around a portion of a limb is provided, and a second surface where a fixing shaft fixing a cuff wrapping around a remaining portion of the limb is mounted; a drive motor coupled to the main body so as to be parallel to the fixing shaft, and driven in response to a control signal received from a controller receiving a signal from a biosignal sensing device or a user input device; a reduction gear unit coupled to a drive shaft of the drive motor to reduce a rotational force of the drive motor; a cuff fixing drum disposed parallel to the drive motor to fix the cuff, and having a first end coupled to the reduction gear unit and a second end rotatably coupled to the main body such that the cuff fixing drum winds and unwinds the cuff in response to the reduced rotational force received from the drive motor; and the cuff including a first end fixed to the cuff fixing drum and a second end wound around and fixed to the fixing shaft via the support portion of the main body so as to wrap around the remaining portion of the limb.

Herein, the main body may include a base frame including the support portion that is provided at a first surface thereof and wraps around the portion of the limb, first and second support frames coupled perpendicular to a second surface of the base frame at positions spaced apart from each other at an interval along a longitudinal direction of the base frame, thus forming a first accommodation space, and a third support frame coupled to the second surface of the base frame at a position spaced apart from the second support frame at an interval, thus forming a second accommodation space.

Herein, the support portion may have a curved surface ergonomically designed to conform to a shape of the limb.

Furthermore, the drive motor may be positioned in the first accommodation space and the drive shaft of the drive motor may be coupled to the second support frame to be positioned in the second accommodation space through the second support frame,

a first side of the reduction gear unit may be coupled to the drive shaft of the drive motor to receive the rotational force from the drive motor and a second side of the reduction gear unit may be coupled to a gear shaft positioned in the second accommodation space to transmit the reduced rotational force of the drive motor, and

a first end of the cuff fixing drum may be coupled to the reduction gear unit through the second support frame and a second end of the cuff fixing drum may be rotatably coupled to the first support frame such that the cuff fixing drum is disposed in the first accommodation space so as to be parallel to the drive motor.

Furthermore, the reduction gear unit may include a first gear coupled to the drive shaft of the drive motor, a second gear engaged with the first gear and coupled to the gear shaft to reduce the rotational force of the drive motor, a third gear coupled to the gear shaft to transmit the rotational force reduced by the second gear, and a fourth gear engaged with the third gear to transmit the reduced rotational force to the cuff fixing drum.

Furthermore, a cuff passing hole through which the cuff fixed to the cuff fixing drum passes may be formed at a first side of the support portion of the main body.

Furthermore, an attaching and detaching member may be provided at the second end of the cuff such that the cuff wound around the fixing shaft may be attachable and detachable, the attaching and detaching member may comprise a male and female pair of Velcro fasteners, and the attaching and detaching member may be configured as multiple attaching and detaching members arranged at a regular interval to allow a length of the cuff to be variable.

Furthermore, a cable entrance port through which a power cable and an encoder signal line connected to the drive motor pass may be provided at the first support frame.

The limb compression device may further include a cover provided at the main body and coupled to the first, second, and third support frames to cover the first and second accommodation spaces to protect the drive motor and the reduction gear unit, wherein the cover may be coupled to the frames, with the fixing shaft exposed externally.

Furthermore, a sound absorbing material may be attached along an inner surface of the cover to absorb noises of the drive motor.

### Advantageous Effects

According to the embodiment of the present invention, it is possible to configure compression intensity personalized to a subject and to realize compression intensity required for treatment. Therefore, the present invention can be used for purposes of treatment (external counterpulsation) and prevention of diseases caused by problems in blood circulation, such as deep vein thrombosis, angina pectoris, heart failure, myocardial infarction, and the like, but also for purposes of massage and vibration by controlling compression intensity, timing, and repetition frequency.

Furthermore, the present invention can realize a compact design through a simplified configuration and an efficient layout structure, thus increasing portability and convenience of use.

Furthermore, the present invention can allow a user to adjust the length of a cuff, thus providing compression personalized to the subject, and to arrange a power cable and an encoder signal line through a cable entrance port, thus increasing practicality and convenience by not restricting a subject's activity.

Furthermore, the present invention can provide a cover mounted to protect a drive motor and a reduction gear unit, thus reducing noises generated from the drive motor.

### DESCRIPTION OF DRAWINGS

FIG. 1 is a perspective view showing a limb compression device according to an embodiment of the present invention viewed from a first direction.

FIG. 2 is a perspective view showing the limb compression device according to the embodiment of the present invention viewed from a second direction.

FIG. 3 is a perspective view showing structure of a main body and arrangement of a drive motor and a reduction gear unit in the limb compression device according to the embodiment of the present invention.

FIGS. 4(a) and 4(b) are exploded perspective views showing configuration of the reduction gear unit in the limb compression device according to the embodiment of the present invention.

FIG. 5 is a main part enlarged view showing a cuff passing hole and a cable entrance port in the limb compression device according to the embodiment of the present invention.

FIG. 6 is a main part enlarged view showing a Velcro fastener provided at a cuff in the limb compression device according to the embodiment of the present invention.

FIG. 7 is a perspective view showing a state in which a cover is further provided in the limb compression device according to the embodiment of the present invention.

### BEST MODE

The above and other objectives, features, and advantages of the present invention will be more clearly understood from the following detailed description taken in conjunction with the accompanying drawings, such that the invention can be easily embodied by one of ordinary skill in the art to which this invention belongs. In the following description, it is to be noted that, when the functions of conventional elements and the detailed description of elements related with the present invention may make the gist of the present invention unclear, a detailed description of those elements will be omitted.

It will be understood that, although the terms first, second, etc. may be used herein to describe various elements, these elements should not be limited by these terms. These terms are only used to distinguish one element, from another element. For instance, a first element discussed below could be termed a second element without departing from the teachings of the present invention. Similarly, the second element could also be termed the first element. The terminology used herein is for the purpose of describing particular embodiments only and is not intended to be limiting of example embodiments of the invention. As used herein, the singular forms “a”, “an”, and “the” are intended to include the plural forms as well, unless the context clearly indicates otherwise.

Although terminologies used in the present specification are selected from general terminologies used currently and widely in consideration of functions, they may be changed in accordance with intentions of technicians engaged in the corresponding fields, customs, advents of new technologies, and the like. Occasionally, some terminologies may be arbitrarily selected by the applicant(s). In this case, the meanings of the arbitrarily selected terminologies shall be described in the corresponding part of the detailed description of the specification. Therefore, terminologies used in the present specification need to be construed on the basis of the substantial meanings of the corresponding terminologies and the overall matters disclosed in the present specification rather than construed as simple names of the terminologies.

Unless the context clearly indicates otherwise, it will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Also, the terms “˜part”, “˜unit”, “module”, “apparatus”, and the like mean a unit for processing at least one function or operation and may be implemented by a combination of hardware and/or software.

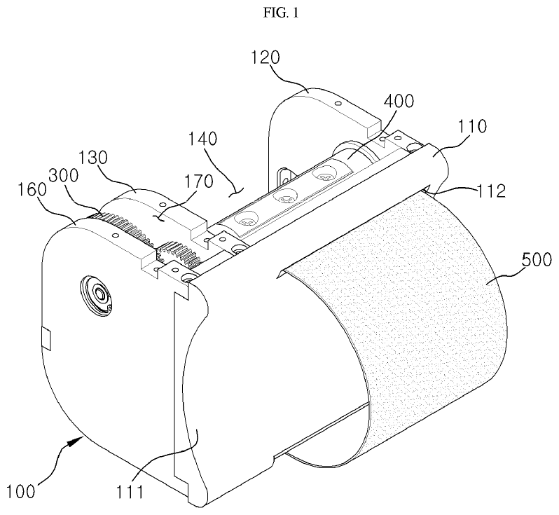

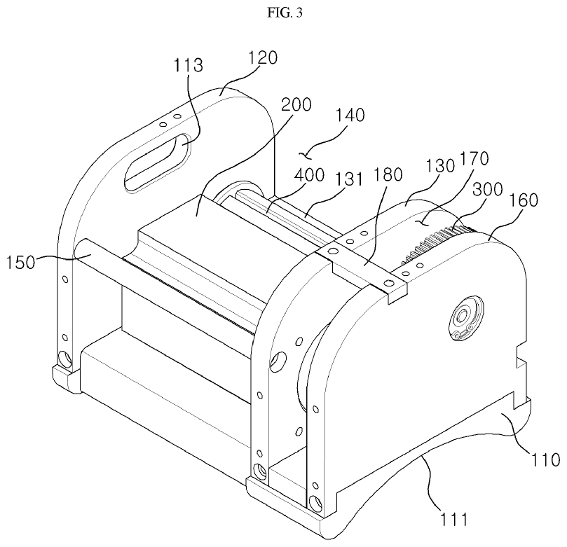

As shown in FIGS. 1 to 3, a limb compression device according to an embodiment of the present invention includes: a main body 100 having a first surface where a support portion wrapping around a portion of a limb is provided, and a second surface where a fixing shaft fixing a cuff wrapping around a remaining portion of the limb is mounted; a drive motor 200 coupled to the main body so as to be parallel to the fixing shaft, and driven in response to a control signal received from a controller receiving a signal from a biosignal sensing device or a user input device; a reduction gear unit 300 coupled to a drive shaft of the drive motor to reduce a rotational force of the drive motor; a cuff fixing drum 400 disposed parallel to the drive motor to fix the cuff, and having a first end coupled to the reduction gear unit and a second end rotatably coupled to the main body such that the cuff fixing drum winds and unwinds the cuff in response to the reduced rotational force received from the drive motor; and the cuff 500 including a first end fixed to the cuff fixing drum and a second end wound around and fixed to the fixing shaft via the support portion of the main body so as to wrap around the remaining portion of the limb.

First, the main body 100 includes a base frame 110 including the support portion 111 provided at a first surface thereof and wrapping around the portion of the limb, and first and second support frames 120 and 130 coupled perpendicular to a second surface of the base frame 100. The first and second support frames 120 and 130 are coupled to the base frame at positions spaced apart from each other at an interval along a longitudinal direction of the base frame 110, thus forming a first accommodation space 140. The fixing shaft 150 provided in the first accommodation space 140 is fixed to first sides of the first and second support frames 120 and 130. The first and second support frames are connected to each other by a reinforcement bar 131.

Furthermore, a third support frame 160 is coupled to the second surface of the base frame 110 at a position spaced apart from the second support frame 130 at an interval, thus forming a second accommodation space 170. The second support frame 130 and the third support frame 160 are connected to each other by a reinforcement bar 180.

Herein, the support portion 111 of the base frame 110 has a curved surface ergonomically designed to conform to the shape of the limb to cover the portion of the limb. The pressure of the cuff 500 surrounding the remaining portion of the limb is applied to the support portion, and thus the support portion applies reaction pressure to the limb, causing the limb to be compressed.

The drive motor 200 is positioned in the first accommodation space 140 formed between the first and second support frames 120 and 130. The drive shaft of the drive motor 200 is coupled to the second support frame 130 to be positioned in the accommodation space 170 through the second support frame 130.

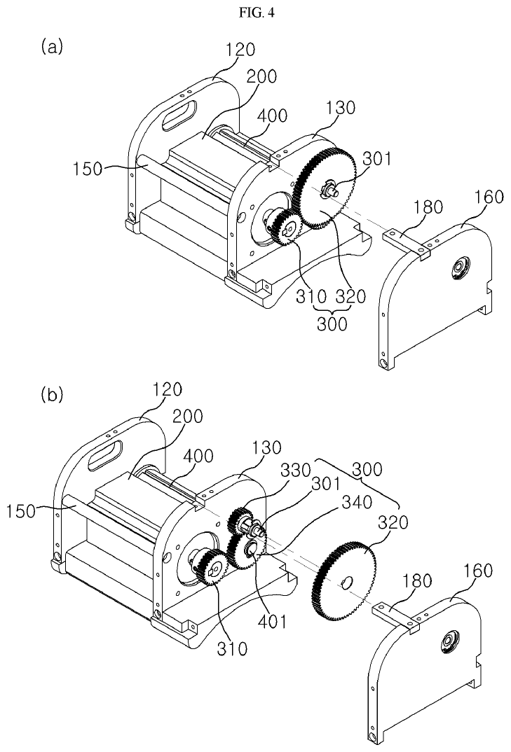

Furthermore, as shown in FIG. 4(a), a first side of the reduction gear unit 300 is coupled to the drive shaft of the drive motor 200 positioned in the second accommodation space 170 through the second support frame 130, such that the reduction gear receives the rotational force from the drive motor 200. A second side of the reduction gear unit is coupled to a gear shaft 301 rotatably coupled to the third support frame 160 to be positioned in the second accommodation space 170, such that the reduction gear unit transmits the reduced rotational force of the drive motor 200.

In more detail, as shown FIG. 4(b), the reduction gear unit 300 includes a first gear 310 coupled to the drive shaft of the drive motor 200, a second gear 320 engaged with the first gear and coupled to the gear shaft to reduce the rotational force of the drive motor, a third gear 330 coupled to the gear shaft 301 to transmit the rotational force reduced by the second gear, and a fourth gear 340 engaged with the third gear and coupled to a cuff fixing drum shaft 401 to transmit the reduced rotational force transmitted from the third gear to the cuff fixing drum 400.

In other words, the first gear 310 coupled to the drive shaft of the drive motor 200 receives the rotational force of the drive motor 200 and transmits the rotational force to the second gear 320 engaged with the first gear, and the second gear 320 reduces the rotational force of the drive motor 200 and transmits the reduced rotational force to the third gear 330 engaged with the second gear. The third gear 330 transmits the reduced rotational force to the fourth gear 340 engaged with the third gear, and the fourth gear 340 transmits the reduced rotational force to the cuff fixing drum 400.

The cuff fixing drum 400 is configured such that a first end of the shaft 401 is coupled to the fourth gear 340 of the reduction gear unit through the second support frame 130 and a second end of the shaft 491 is rotatably coupled to the first support frame 120. The cuff fixing drum is disposed in the first accommodation space 140, in parallel arrangement to the drive motor 200.

Herein, the first end of the cuff 500 is fixed to the cuff fixing drum 400 by means of bolt fastening.

Therefore, the cuff fixing drum 400 is rotated in response to the reduced rotational force received from the reduction gear unit 300 when the drive motor 200 is rotated, thus winding and unwinding the cuff 500.

Herein, the reduction gear unit 300 provides effects of reduced speed and increased torque. In addition, due to the shape of being accommodated in the second accommodation space, there is provided an effect of replacing a conventional motor gearbox.

In addition, with the structure of the reduction gear unit 300, the cuff fixing drum 400 has an arrangement structure parallel to the drive motor 200. This realizes a compact design, leading to increasing portability and convenience of use.

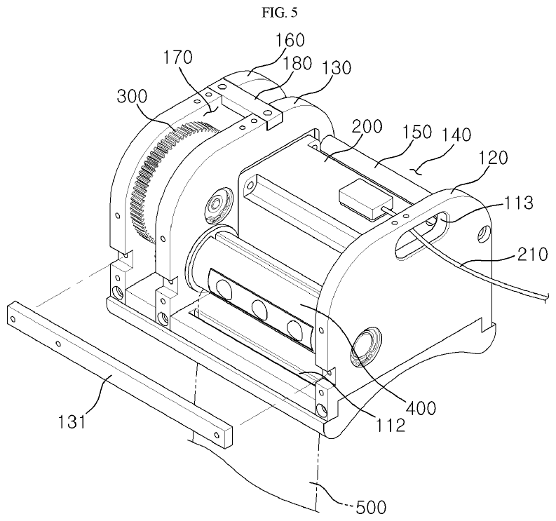

Meanwhile, as shown in FIG. 5, the cuff passing hole 112 through which the cuff 500 fixed to the cuff fixing drum 400 passes is formed at a first side of the support portion 111 of the main body 100.

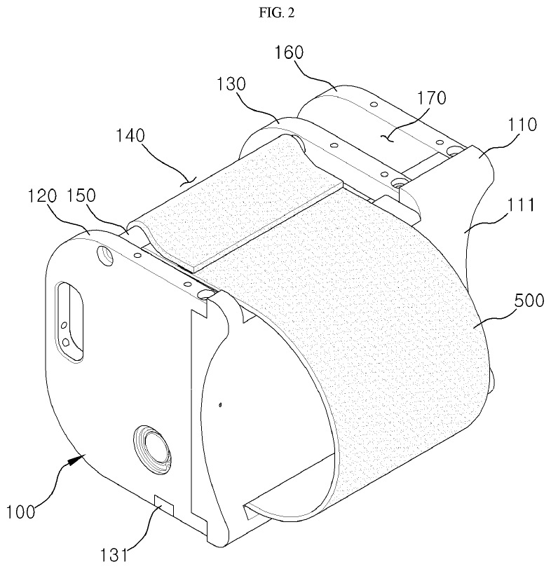

As shown in FIGS. 1 and 2, the cuff 500 having the first end fixed to the cuff fixing drum 400 is wound around the fixing shaft 150 via the support portion 111 through the cuff passing hole 112.

In other words, the cuff 500 wraps around the limb positioned between the support portion 111 and the cuff 500 in a round or oval shape while facing the support portion 111. The cuff compresses the limb according to the degree of winding by the cuff fixing drum 400.

Herein, a male and female pair of attaching and detaching members 510 is provided at the second end of the cuff 500 such that the cuff 500 wound around the fixing shaft 150 is attachable and detachable. Herein, examples of the attaching and detaching members 510 include a male and female pair of Velcro fasteners and snap buttons.

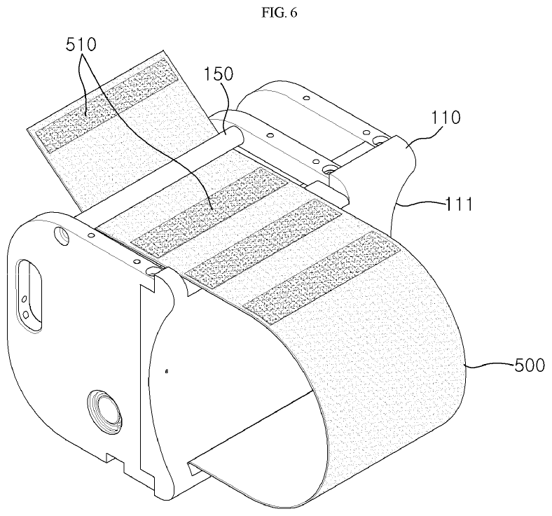

In addition, multiple attachable and attaching and detaching members 510 may be provided at a regular interval to allow the length of the cuff to be variable. As an example, as shown in FIG. 6, a male Velcro fastener may be attached to the second end of the cuff 500, and multiple female Velcro fasteners may be attached at a regular interval at positions overlapping with the second end of the cuff wound around the fixing shaft 150. In this case, the length of the cuff can be adjusted according to the position where the male Velcro Fastener is selectively attached to the female Velcro fasteners.

Meanwhile, as shown in FIG. 5, a cable entrance port 113 through which a power cable and an encoder signal line 210 connected to the drive motor 200 pass is provided at the first support frame 120 of the main body 100.

The cable entrance port 113 provides a space for arranging the power cable and the encoder signal line 210. This improves convenience of use by not restricting a wearer's activity.

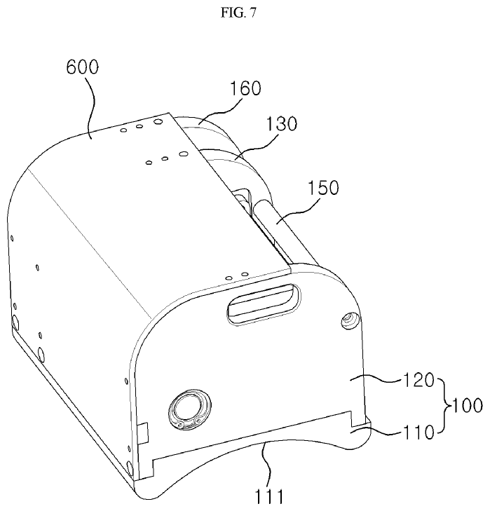

In addition, as shown in FIG. 7, the present invention may further include a cover 600 provided at the main body 100 so as to cover the first and second accommodation spaces to protect the drive motor 200 and the reduction gear unit 300. Herein, the cover 600 is coupled to the first, second, and third support frames 120, 130, and 160 to cover the first and second accommodation spaces 140 and 170, with the fixing shaft 150 exposed externally to facilitate attachment and detachment of the cuff 500.

In addition, a sound absorbing material is attached to an inner surface of the cover 600 to absorb and reduce noises generated from the drive motor 200.

The operation of the limb compression device configured as described above will be described. The limb is positioned on the support portion 111 of the main body 100, the cuff 500 fixed to the cuff fixing drum 400 is unwound and extended to wrap around the limb, and the second end of the cuff 500 wound around the fixing shaft 150 to attach the female and male Velcro fasteners to each other at positions overlapping each other.

Subsequently, when power is applied, the drive motor 200 is driven in response to the control signal of the controller receiving the signal from the biosignal sensing device or the user input device.

Herein, the biosignal sensing device is a device for measuring biosignals of a subject and may include measuring means for measuring various biosignals, such as electrocardiogram measuring means, blood flow measuring means, blood pressure measuring means, body temperature measuring means, skin resistance measuring means, and the like. The measured biosignals are transmitted to the controller. The configuration of the biosignal sensing device is a general known technique, and a detailed description thereof will be omitted.

In addition, the user input device may include an input key implemented in various ways, such as a key button, a touch pad, or the like, to receive an operator or subject's intention. The input key includes a compression intensity setting key, a timing setting key, and a repetition frequency setting key. There may be further included an input key for which various functions are set, such as a function set for treatment of diseases caused by problems in blood circulation, including deep vein thrombosis, angina pectoris, heart failure, and myocardial infarction, a massage function, a vibrating function, and the like. The signal input through the input key is transmitted to the controller.

Subsequently, the rotational force of the drive motor 200 driven in response to the control signal of the controller is reduced by the reduction gear unit 300 and transmitted to the cuff fixing drum 400. The cuff fixing drum 400 is rotated in response to the reduced rotational force, causing the cuff 500 to be wound to compress the limb. Herein, the drive motor 200 repeats normal and reverse rotations in response to the control signal of the controller, causing the cuff 500 to be wound and unwound to repeatedly compress and decompress the limb.

As described above, the present invention is characterized by compressing the limb by winding the cuff by means of rotation of the cuff fixing drum other than inflating the cuff by means of pneumatic pressure, thus realizing compression intensity required for treatment. In addition, the present invention can be used not only for the purposes of treatment and prevention of diseases caused by problems in blood circulation, such as deep vein thrombosis, angina pectoris, heart failure, myocardial infarction, and the like that require compression intensity, but also for the purposes of massage and vibration by controlling compression intensity, timing, and repetition frequency.

Furthermore, the present invention is characterized by realizing a compact design through a simplified configuration and an efficient layout structure, thus increasing portability and convenience of use.

Furthermore, the present invention is characterized by adjusting the length of the cuff, thus providing compression personalized to the subject, and by arranging the power cable and the encoder signal line through the cable entrance port, thus increasing practicality and convenience by not restricting a subject's activity.

Furthermore, the present invention is characterized by the provision of the cover mounted to protect the drive motor and the reduction gear unit, thus reducing noises generated from the drive motor.

Although an embodiment of the present invention has been described in detail above, the scope of the present invention is not limited to the embodiment. It will be understood by those of ordinary skill in the art that various changes and modifications may be made therein without departing from the technical idea and scope of the present invention and such changes, modifications, and all equivalents belong to the scope of the present invention.

### INDUSTRIAL APPLICABILITY

The present invention can find wide application in an external counterpulsation system.

1. A limb compression device, comprising:
a main body having a first surface where a support portion wrapping around a portion of a limb is provided, and a second surface where a fixing shaft fixing a cuff wrapping around a remaining portion of the limb is mounted;
a drive motor coupled to the main body so as to be parallel to the fixing shaft, and driven in response to a control signal received from a controller receiving a signal from a biosignal sensing device or a user input device;
a reduction gear unit coupled to a drive shaft of the drive motor to reduce a rotational force of the drive motor;
a cuff fixing drum disposed parallel to the drive motor to fix the cuff, and having a first end coupled to the reduction gear unit and a second end rotatably coupled to the main body such that the cuff fixing drum winds and unwinds the cuff in response to the reduced rotational force received from the drive motor; and
the cuff including a first end fixed to the cuff fixing drum and a second end wound around and fixed to the fixing shaft via the support portion of the main body so as to wrap around the remaining portion of the limb.

2. The limb compression device of claim 1, wherein the main body includes
a base frame including the support portion that is provided at a first surface thereof and wraps around the portion of the limb,
first and second support frames coupled perpendicular to a second surface of the base frame at positions spaced apart from each other at an interval along a longitudinal direction of the base frame, thus forming a first accommodation space, and
a third support frame coupled to the second surface of the base frame at a position spaced apart from the second support frame at an interval, thus forming a second accommodation space.

3. The limb compression device of claim 2, wherein the support portion has a curved surface ergonomically designed to conform to a shape of the limb.

4. The limb compression device of claim 2, wherein the drive motor is positioned in the first accommodation space and the drive shaft of the drive motor is coupled to the second support frame to be positioned in the second accommodation space through the second support frame,
a first side of the reduction gear unit is coupled to the drive shaft of the drive motor to receive the rotational force from the drive motor and a second side of the reduction gear unit is coupled to a gear shaft positioned in the second accommodation space to transmit the reduced rotational force of the drive motor, and
a first end of the cuff fixing drum is coupled to the reduction gear unit through the second support frame and a second end of the cuff fixing drum is rotatably coupled to the first support frame such that the cuff fixing drum is disposed in the first accommodation space so as to be parallel to the drive motor.

5. The limb compression device of claim 4, wherein the reduction gear unit includes
a first gear coupled to the drive shaft of the drive motor,
a second gear engaged with the first gear and coupled to the gear shaft to reduce the rotational force of the drive motor,
a third gear coupled to the gear shaft to transmit the rotational force reduced by the second gear, and
a fourth gear engaged with the third gear to transmit the reduced rotational force to the cuff fixing drum.

6. The limb compression device of claim 1, wherein a cuff passing hole through which the cuff fixed to the cuff fixing drum passes is formed at a first side of the support portion of the main body.

7. The limb compression device of claim 1, wherein an attaching and detaching member is provided at the second end of the cuff such that the cuff wound around the fixing shaft is attachable and detachable.

8. The limb compression device of claim 7, wherein the attaching and detaching member comprises a male and female pair of Velcro fasteners.

9. The limb compression device of claim 7, wherein the attaching and detaching member is configured as multiple attaching and detaching members arranged at a regular interval to allow a length of the cuff to be variable.

10. The limb compression device of claim 4, wherein a cable entrance port through which a power cable and an encoder signal line connected to the drive motor pass is provided at the first support frame.

11. The limb compression device of claim 4, further comprising: a cover provided at the main body and coupled to the first, second, and third support frames to cover the first and second accommodation spaces to protect the drive motor and the reduction gear unit, wherein the cover is coupled to the frames, with the fixing shaft exposed externally.

12. The limb compression device of claim 11, wherein a sound absorbing material is attached along an inner surface of the cover to absorb noises of the drive motor.
